(19) United States
(12) Patent Application Publication
(10) Pub. No.: US 2019/0255115 A1
(43) Pub. Date: Aug. 22, 2019

(54) USE OF MESENCHYMAL STEM CELLS AND PARTS THEREOF

(71) Applicant: Erasmus University Medical Center Rotterdam, Rotterdam (NL)

(72) Inventor: Martin J. Hoogduijn, Rotterdam (NL)

(21) Appl. No.: 16/304,514

(22) PCT Filed: May 26, 2017
(86) PCT No.: PCT/NL2017/050334

§ 371 Date: Nov. 26, 2018

(30) Foreign Application Priority Data
May 25, 2016 (EP) 16171332.6

(51) Int. Cl. A61K 35/28; C12N 5/0775; A61P 37/06

(52) U.S. Cl. CPC A61K 35/28; A61K 2035/122; A61P 37/06; C12N 5/0667

(57) ABSTRACT

The invention relates to immunomodulatory particles from lysed mesenchymal stem cells, comprising membranous structures from said mesenchymal stem cells, and to their use as a medicament. Said medicament preferably is for the treatment of acute and chronic inflammatory diseases and of autoimmune diseases. The invention further relates to a pharmaceutical composition comprising the immunomodulatory particles, and to inactivated mesenchymal stem cells, or parts thereof, for use as a medicament.

### FIELD OF THE INVENTION

The invention relates to mesenchymal stem cells and parts thereof and their use in immunomodulatory therapies.

Multipotent Mesenchymal stem cells (MSC) are present in most adult human tissues and can be easily obtained from adipose tissue and bone marrow. MSC are characterized by their ability to proliferate in a plastic-adherent manner and have the capacity to differentiate into osteocytes, adipocytes, myocytes and chondrocytes (Pittenger et al., 1999. Science 284: 143-147). In addition, MSC possess immunosuppressive properties as demonstrated in experimental inflammatory disease models such as, for instance, autoimmune diseases, graft-versus-host disease (GvHD) and allograft rejection (Gonzalez et al., 2009. Gastroenterology 136: 978-989; Constantin et al., 2009. Stem Cells 27: 2624-2635; Popp et al., 2008. Transpl Immunol 20: 55-60; Roemeling-van Rhijn et al., 2013. J Stem Cell Res Ther Suppl 6: 20780; Gonzalez-Rey et al., 2009. Gut 58: 929-939; Augello et al., 2007. Arthritis Rheum 56: 1175-1186; Tobin et al., 2013. Clin Exp Immunol 172: 333-348; Joo et al., 2010. Cytotherapy 12: 361-370). The promising results obtained from these models have triggered the investigation of MSC therapy in clinical trials for a range of immune disorders, including GvHD, Crohn's disease, Diabetes mellitus, Systemic Lupus Erythematosus (SLE) and to prevent allograft rejection (Le Blanc et al., 2008. Lancet 371: 1579-86; Bernardo et al., 2011. Bone Marrow Transplant 46: 200-207; Hu et al., 2013. Endocr J 60: 347-357; Forbes et al., 2014. Clin Gastroenterol Hepatol 12: 64-71; Wang et al., 2013. Cell Transplant 22: 2267-2277).

Whereas some randomized clinical trials describe a positive effect of MSC treatment, other studies do not show amelioration of disease symptoms after MSC treatment (Luk et al., 2015. Expert Rev Clin Immunol 11: 617-636). The indistinct efficacy of MSC immunotherapy is debit to a lack of understanding of the mechanisms of immunomodulation by MSC after in vivo administration, which hampers rational timing and dosing of MSC therapy and hinders distinction between conditions that can potentially benefit from MSC therapy and conditions that cannot.

In vitro studies show that under the influence of an inflammatory environment MSC inhibit the proliferation of immune cells via soluble mechanisms such as TGF-6, prostaglandin E2 (PGE2) and indolamine 2,3 dioxygenase (IDO) (Waterman et al., 2010. PLoS One 5: e10088; Di Nicola et al., 2002. Blood 99: 3838-3843; Groh et al., 2005. Exp Hematol 33: 928-934; Spaggiari et al., 2008. Blood 111: 1327-1133; Hsu et al., 2013. J Immunol 190: 2372-2380; Liang et al., 2013. Zhonghua Xueyexue Zazhi 34: 213-216; Gu et al., 2013. Hum Immunol 74: 267-276; Luz-Crawford et al., 2012. PLoS One 7: e45272). Furthermore, it has been reported that immune-regulatory factors of MSC may be enriched in small extracellular vesicles such as exosomes and microvesicles that are released from the plasma membrane, endoplasmic reticulum or endosomes of living MSC (Kordelas et al., 2014. Leukemia 28: 970-973). These findings seem to be confirmed in a recent patent application, PCT/IT2012/000232, which reports that microvesicles isolated from living mesenchymal stem cells can be used as immunosuppressive agents for treatment of inflammatory and immune pathologies. Said microvesicles were found to modulate the function of multiple immune cell types (Di Trapani et al., 2016. Sci Rep. 13; 6: 24120). These results suggest that the immunomodulatory effects of MSC may be mediated by microvesicles that are actively released by living MSC.

It is therefore proposed that the therapeutic immunomodulatory effects of MSC are mediated via their secretome (Caplan and Correa, 2011. Cell Stem Cell 9: 11-15). However, there is no crystal clear evidence that the secretome of MSC is responsible for their immunomodulatory effects after in vivo administration. MSC get trapped in the small capillaries of the lungs after intravenous (i.v.) administration and the majority of MSC die within 24 hours after infusion (Eggenhofer et al., 2012. Front Immunol 3: 297; Schrepfer et al., 2007. Transplant Proc 39: 573-576). This raises the questions whether MSC live long enough after i.v. infusion to become activated by inflammatory conditions and exert their therapeutic effects via their secretome or whether they can escape the lung capillaries and migrate to sites of inflammation.

It has become clear that MSC, rather than having direct immunomodulatory effects on target cells, exert at least some of their effects after infusion via activation of recipient cells. For example, it has been shown that the protective effect of MSC on cardiac infarct repair is partially mediated by modulation of reparative M2 macrophages since early macrophage depletion partially reduced the therapeutic effect of MSC (Ben-Mordechai et al., 2013. J Am Coll Cardiol 62: 1890-1901). It was recently demonstrated that infusion of MSC triggers a mild systemic inflammatory response, which may be the initiator of subsequent immunosuppression (Hoogduijn et al., 2013. Stem Cells Dev 22: 2825-2835). Whether or not the secretome is required for achieving this inflammatory response is presently not known.

### BRIEF DESCRIPTION OF THE INVENTION

It is an object of the present invention to explore whether the immunomodulatory effects of MSC are mediated only by living MSC that actively interact with immune cells and release cytokines, growth factors and vesicles. The presented studies surprisingly show that MSC also trigger immunomodulatory responses of host cells via passive mechanisms.

The invention is therefore directed to immunomodulatory membranous particles from lysed MSC comprising membranous structures from said MSC.

The invention is based on the surprising finding that inactivated MSC that are secretome deficient are able to modulate the immune system of a subject, after administration of the inactivated MSC to the subject. Thus far, it was generally believed that the beneficial effects of MSC are mediated by actively secreted immune response-modulating factors.

It is now surprisingly found that some immune-regulatory properties of MSC, including the induction of differentiation of monocytes can be mediated by immunomodulatory particles from lysed mesenchymal stem cells comprising membranous structures from said mesenchymal stem cells. Importantly, the immunomodulatory particles from lysed MSC do not directly inhibit T-cell proliferation and/or do not directly modulate B-cell functions. Therefore, the immunomodulatory membranous particles differ also in this respect from small extracellular vesicles that are released from the plasma membrane of living MSC.

Said immunomodulatory particles preferably have an average particle size of between 70 and 170 nm, preferably between 90 and 150 nm, more preferably about 120 nm.

The use of the immunomodulatory particles from lysed MSC will strongly reduce a risk of transmission of pathogens such as viruses, that is associated with the administration of live MSC to a subject.

The particles of the invention are preferably generated from MSC that have been treated with interferon gamma, prior to their lysis. Pretreatment of MSC with cytokines such as interferon gamma was found to trigger the immunosuppressive function of MSC, and also the immunomodulatory function of membranous particles derived from lysed MSC that had been pre-treated with interferon gamma, when compared to MSC that were not pre-treated.

The particles according the invention preferably are for use as a medicament, preferably in the treatment of acute and chronic inflammatory diseases and of autoimmune diseases, or in the treatment and prevention of transplant rejection

MSC are low immunogenic. Therefore, the immunomodulatory membranous particles are preferably prepared from allogenic MSC, i.e. from one or more subjects of the same species, preferably from one or more human subjects. To further prevent an immune response against particles of the invention after administration to a subject, the particles may be prepared from MSC that are obtained from a subject to be treated with said particles.

The invention further provides a pharmaceutical composition comprising the immunomodulatory particles from lysed mesenchymal stem cells comprising membranous structures from said mesenchymal stem cells, and a pharmaceutically acceptable excipient.

Said pharmaceutical composition preferably is for use in immunosuppressive therapy and/or for use in the treatment and prevention of transplant rejection.

The invention further provides inactivated MSC, or parts thereof, for use as a medicament, preferably for use in the treatment of acute and chronic inflammatory diseases, including the treatment of autoimmune diseases, and or the treatment and prevention of transplant rejection.

### FIGURE LEGENDS

FIG. 1. Shape and size characteristics of MSC particles. A: Confocal microscopy image showing the round structures of the membranous particles stained with fluorescent PKH26, shown in grayscale. B: Size distribution of the particles derived from MSC and from IFNγ-treated MSC measured by Nanosight showing a size range between 70 nm and 600 nm with a peak at 100-120 nm.

FIG. 2. Flow cytometric analysis of MSC and MSC particles. MSC show expression of CD73 and CD90 but have very low levels of PDL1 (left column). Treatment of MSC with IFNγ preserves CD73 and CD90 expression and upregulates PDL1 expression. The immunophenotype of MSC particles mimics the immunophenotype of MSC, with expression of CD73 and CD90 in particles derived from MSC and from IFNγ treated MSC, and PDL1 expression only in particles from IFNγ treated MSC (right column).

FIG. 3. MSC particles affect immunophenotype of human CD14⁺ monocytes isolated from peripheral blood. A: Addition of MSC particles to monocytes for 24 h has a dose-dependent effect on CD90 expression on monocytes. B: Particles from MSC treated with IFNγ, but not from control MSC, dose-dependently increase anti-inflammatory PD-L1 expression on monocytes. * indicates statistical significance compared to no particles.

FIG. 4. MSC particles affect cytokine mRNA expression of human CD14⁺ monocytes isolated from peripheral blood. A: CD14⁺ monocytes increased the expression of IL6 upon culture in the presence of MSC particles for 24 h. B: CD14⁺ monocytes increased the expression of IL10 upon culture in the presence of MSC particles for 24 h. There were no differences in the effects of particles derived from control MSC or IFNγ treated MSC. Particles were added at a 1:40,000 ratio to CD14⁺ monocytes.

FIG. 5. Infusion of MSC particles in mice affects systemic cytokine and chemokine levels. C57BL6 mice received 5 mg/kg LPS to induce a systemic inflammatory response and MSC particles (10×10⁹) were administered intravenously after 1 hour. Six hours after LPS administration blood was analysed for cytokine and chemokine levels by milliplex assay. A: MSC particles and MSC(IFNγ) particles increased serum G-CSF levels and B: MIP1α levels. C: MSC(IFNγ) particles only increased IL10 levels.

FIG. 6. MP characterization. Morphological characterization of MP generated from unstimulated and IFN-γ MSC (MP and MPγ, respectively). (A) Size distribution of MP and MPγ measured by NTA. (B) The average number of particles generated per MSC. (C) Transmission electron microscopy analysis of MP.

FIG. 7. Enzymatic activity of MP. (A) ATPase activity was measured at four different concentrations of MP (1×10¹², 1×10¹¹, 1×10¹⁰, 1×10⁹/ml). MP and MPγ were able to catalyze the reaction and the detection of free phosphate was dependent on concentration of MP. (B) The activity of CD73 was measured for three different concentrations of MP (1×10¹², 1×10¹¹, 1×10¹⁰/ml). MP and MPγ were able to produce free phosphates after adding the substrate (AMP) and it was dependent on the concentration of MP. CD73 enzyme (2 and 1 ng) was used to relative calculate the concentration of CD73 in the MP. (C) The esterase activity of three different concentrations of MP (1×10⁹, 1×10⁸, 1×10⁷ particles/ml) was measured by the conversion of CFDA-SE to CFSE by flow cytometry. Fluorescent events were observed in MP labeled with CFSE (CFSE-MP), and the number of CFSE-MP detected was dependent on the concentration of MP. There was no statistical difference between MP, and MPγ in the experiments.

FIG. 8. Effect of MP on CD14⁺ cells. Monocytes were cultured with different ratios of MP for 24 h (1:10,000, 1:40,000, 1:80,000) to determine the effect of MPs on monocyte immunophenotype. (A) Expression of CD16 molecules on monocytes cultured in the presence of MP (n=6; mean±SEM). (B and C) Expression of CD90 and PD-L1 in CD14⁺ CD16⁺ monocytes in the presence of MP (n=7; mean±SEM). (D) mRNA expression of monocytes after culture with MP. After 24 h of culture with MP, monocytes were separated from MP and assessed by real-time PCR and the assay-on-demand primer/probes for CD90, IDO, PD-L1, IL-6, TNF-α and IL-10. (n=6; mean±SEM). Paired T test, *p<0.05, **p<0.01 and ***p<0.001 vs control; #p<0.05 and ##p<0.01 vs MP group.

FIG. 9. Uptake of MP by monocytes. PKH-MP were added to PBMC (ratio 1:40,000) and incubated during 1 h, and 24 h at 37° C. As control the experiment was incubated at 4° C. (A and B) PKH-MP uptake by lymphocytes (CD3) and monocytes (CD14) was analyzed by flow cytometry.

FIG. 10. Immunofluorescence analysis of MP uptake by monocytes. Confocal microscopy analysis of PKH-MP uptake by monocytes. (A) Time-lapse recordings showed that the MP bound to the plasma membrane of the monocytes but they were not internalized. (B) Z-stack images of the MP co-localization on the monocytes.

### DESCRIPTION

### Definitions

The term “Mesenchymal Stem Cells” or MSC, as is used herein, refers to adult progenitor cells that can self-renew and can differentiate into multiple lineages such as osteoblasts, adipocytes and chondroblasts. MSC can be isolated from numerous tissues such as bone marrow, adipose tissue, the umbilical cord, liver, muscle, and lung. MSC adhere to plastic when maintained under standard culture conditions. MSC express CD73, CD90 and CD105, but under standard culture conditions lack expression of CD45, CD11b, CD19 and HLA-DR surface molecules.

The term “membranous particles”, as is used herein, refers to plasma membrane fragments that are generated upon lysis of cells. The term “membranous particles” is explicitly used to differentiate these particles from naturally occurring extracellular microvesicles, which include exosomes, which are small intracellularly-generated vesicles, and vesicles that are naturally shed from the cell membrane of living cells. Said membranous particles express CD73, which is absent from, for example, exosomes. In addition, whereas naturally shedded vesicles such as extracellular vesicles are highly enriched in tetraspanins such as CD63 and CD81, these tetraspanins are not enriched on membranous particles. A level expression of tetraspanins such as CD63 and CD81 on membranous particles is similar to the level of expression on the plasma membrane. Said level of expression is at most 20%, more preferred at most 10% of the level of expression on naturally shedded vesicles such as extracellular vesicles. The term “immunomodulatory”, as is used herein, refers to the ability to alter an immune response. A preferred immunomodulatory activity is suppression of an immune-related disease such as graft-versus-host disease, auto-immune disease and an inflammatory disease such as Crohn's disease. It can also refer to activation of the immune system in situations where immune activity is insufficient to fight infections or when the recovery of the immune system after ablation is impaired.

### Immunomodulatory Membranous Particles

Mesenchymal stem cells may be isolated by enzymatic treatment, preferably collagenase treatment, of tissue such as bone marrow or adipose tissue, as is known to the skilled person. Density fractionation may be employed to separate mononuclear cells from erythrocytes and granulocytes. As an alternative, red blood cell lysis may be used for the isolation of human MSC from bone marrow aspirate (Francis et al., 2010. Organogenesis 6: 11-14). Plating of cells on plastic and selection of cells that adhere to plastic preferably is used in the isolation procedure of MSC. In addition, sorting techniques including magnetic bead coupling may be performed to enrich MSC, for example to remove contaminating cells such as CD45+ cells.

In a preferred method, adipose tissue is enzymatically digested with collagenase type IV at 37° C. under continuous shaking. After centrifugation, the cell pellet is resuspended and incubated at room temperature. The cells are then washed, resuspended in MEM-α supplemented with 2 mM L-glutamine, 1% penicillin/streptavidine (p/s), and 15% fetal bovine serum (FBS) in a humidified atmosphere with 5% CO2 at 37° C. Non-adherent cells are subsequently removed after 3-4 days.

Isolated MSC may be lysed by any method known in the art, including mechanical lysis and/or the addition of a lysis buffer. Said lysis buffer preferably controls ionic strength and/or osmotic strength. Chaotropic agents such as chloride or isothiocyanate may be added to enhance lysis of MSC. Said lysis buffer preferably does not comprise a detergent such as Triton X-100 or SDS.

Lysis of MSC preferably is performed by incubation in a hypotonic lysis buffer and application of mechanical disruption, for example by a Dounce homogenizer or a Potter-Elvehjem homogenizer. As an alternative, the cell may be lysed by freeze-thawing. A most preferred lysis buffer is a hypotonic lysis buffer. Said hypotonic lysis buffer preferably is water.

The membrane fraction of lysed cells preferably is recovered by centrifugation, preferably ultracentrifugation, preferably by centrifuging for 20 minutes at 100,000×g. Organelles may be washed off with a buffer, such as phosphate-buffered saline (PBS), hepes-buffered solution (HBS) or MES-buffered solution (MBS). The resulting membrane parts are considered to re-anneal to generate the membranous particles. These particles are structures with smaller diameters which preserve MSCs surface proteins.

A person skilled in the art will understand that molecules can be added during re-annealing of the membranous particles. Those molecules preferably are immune-modulating compounds, preferably immune suppressive compounds. In this way, said immunosuppressive compounds will be included in the membranous particles. Examples of such compounds are steroids, preferably glucocorticoids such as hydrocortisone, cortisone, prednisone, prednisolon and dexamethason, cytostatics, antibodies, and calcineurin inhibitors such as cyclosporin and tacrolimus. Hence, the invention also provides immunomodulatory particles comprising membranous structures from the plasma membrane of said mesenchymal stem cells, said membraneous structures comprising immunosuppressive compounds, preferably steroids, cytostatics, antibodies, and/or calcineurin inhibitors.

The average particle size of the resulting membranous particles may be determined by dynamic light scattering, scanning electron microscopy, size exclusion chromatography, gel electrophoresis, asymmetrical flow field-flow fractionation, analytical ultracentrifugation or, preferably by Nanoparticle Tracking Analysis (Malvern, Enigma Business Park, Malvern, WR14 1XZ, United Kingdom). The membranous particles according to the invention have an average particle size of between 70 and 170 nm, preferably between 90 and 150 nm, more preferably about 120 nm. For comparison, microvesicles have an average particle size of 50-1000 nm, but are generally larger than 250 nm. Exosomes have an average particle size of 30-100 nm. The small size of the membranous particles, when compared to MSC, renders the membranous particles potentially more efficient for immunomodulation in systemic immune diseases, such as graft versus host disease and sepsis because of their better systemic distribution. Furthermore, the membranous particles may be efficient in localised immune disorders as they are able to pass capillary networks and reach inflamed sites. In addition, the membranous particles are easier to generate in large numbers needed for clinical application than naturally secreted vesicles. A further advantage of membranous particles, when compared to intact MSC, is that membranous particles are non-tumorigenic and probably will not transmit pathogenic agents such as viruses.

The state or quality of the membranous particles is preferably determined before their subsequent use in immunomodulatory therapy. A preferred assay to determine the quality of the membranous particles is an ATPase assay. ATP cleavage by membranous particles is linked to substrate translocation over the membrane, as the energy for substrate translocation is derived from ATP hydrolysis. ATP hydrolysis yields inorganic phosphate, which can be measured by a simple colorimetric reaction. The amount of liberated inorganic phosphate is directly proportional to the ATPase activity. Said ATPase assay is preferably determined as described in Meshcheryakov and Wolf., 2016. Protein Science doi.org/10.1002/pro.2932.

A threshold for membranous particles of sufficient quality is an ATPase activity that converts at least 0.1, 0.5, 1, 5 or, preferably, at least 10 μM of ATP per 2.5×10⁷ membranous particles in 30 minutes.

The isolated MSC preferably are pretreated prior to isolating membranous particles to increase the immunosuppressive potential of the MSC. Pre-treatment preferably is performed by culturing the cells for 1-10 days, preferably about 3 days, with one or more cytokines Preferred cytokines include tumor necrosis factor alpha, interleukin 1 alpha, interleukin 1 beta, transforming growth factor beta and interferon gamma, or combinations thereof. A preferred cytokine is interferon gamma, or a combination of interferon gamma with one or more of tumor necrosis factor alpha, interleukin 1 alpha, interleukin 1 beta, and transforming growth factor beta.

MSC are preferably pre-treated with cytokines for a period of 2-5 days, preferably about 3 days, prior to their lysis. Pretreatment preferably includes incubation of the cells with 50 ng/ml IFN-γ. It was found that immunomodulatory proteins on MSC become upregulated after pre-treatment with IFN-γ, amongst them programmed death ligand 1 (PDL1).

The MSC may be inactivated prior to their lysis. Inactivation may occur by any mechanism known in the art, including heat treatment, radiation such as ultra-violet radiation and ionizing radiation such as X-ray radiation, and/or chemical treatment. MSC are preferably inactivated by heat treatment, preferably by incubation in a temperature-regulated water bath at 45-55° C., preferably at about 50° C., preferably for a period from 10 minutes to 1 hour, preferably for a period of about 30 minutes.

### Immunomodulatory Membranous Particles as Medicament

The invention further provides membranous particles according to the invention for use as a medicament. Said membranous particles may be administered to a subject in need thereof by parenteral administration or by nasal and/or intratracheal administration, for example through inhalation or through the use of nose-sprays. Parenteral administration refers to a route of administration which is selected from intravenous, intra-arterial, intramuscular, subcutaneous, intradermal, and intraperitoneal administration. Preferred administration routes are intravenous administration and intra-arterial administration, preferably intravenous or intra-arterial injection or intravenous or intra-arterial perfusion.

Said membranous particles preferably are dosed at 10E7-10E13 membranous particles per kilogram bodyweight of a receiving subject, preferably at 10E8-10E12 membranous particles per kilogram bodyweight, 10E9-10E11 membranous particles per kilogram bodyweight, more preferably at about 10E10 membranous particles per kilogram bodyweight.

Said membranous particles preferably are provided as an aqueous suspension, more preferably as an isotonic aqueous suspension.

Said membranous particles are preferably for use as a medicament in the treatment of acute and chronic inflammatory diseases, including autoimmune diseases. Said membranous particles may also be used as a medicament in the treatment of multiple system atrophy, multiple sclerosis, amyotrophic lateral sclerosis, and stroke.

Examples of inflammatory diseases that may be treated with the membranous particles according to the invention include acne, Addison's disease, asthma, celiac disease, prostatitis, glomerulonephritis, graft-versus-host disease, Hashimoto's disease, interstitial cystitis, lupus erythematosus, inflammatory bowel diseases such as Crohn's disease, pelvic inflammatory disease, psoriasis, rheumatoid arthritis, sarcoidosis, scleroderma, sepsis, Sjögren's syndrome, type 1 diabetes, transplant rejection, and vasculitis.

Said membranous particles are preferably for use as a medicament in the treatment and prevention of transplant rejection. Transplant rejection is mediated by an adaptive immune response via cellular immunity and humoral immunity. Transplant rejection may be acute, occurring from the first week after the transplant to 3 months afterward; or chronic, occurring over many years. The membranous particles provide an immunomodulatory and pro-tolerogenic tool in, during or after organ transplantation and could substitute or minimize current immunosuppressive treatments, which come with major side effects.

Said membranous particles may be combined with one or more immunosuppressive agents that are used in organ transplantation, such as corticosteroids such as prednisone or methylprednisolone, calcineurin inhibitors such as cyclosporine and tacrolimus, antiproliferative agents such as mycophenolate mofetil, azathioprine, or sirolimus, monoclonal antilymphocyte antibodies such as muromonab-CD3, interleukin-2 receptor antagonist, or daclizumab and/or polyclonal antilymphocyte antibodies such as antithymocyte globulin-equine or antithymocyte globulin-rabbit in the treatment and prevention of transplant rejection.

The membranous particles for use as a medicament can be generated from autologous and allogeneic MSC. Although MSC are low immunogenic, allowing the use of allogenic MSC for the preparation of the membranous particles, the membranous particles may be obtained from MSC of a subject to be treated with said particles. The use of particles from autologous cells may have therapeutic applications in autoimmune diseases or pathologies that allow enough time for isolation and in vitro expansion of MSC. However, the clinical applications performed to date with allogeneic MSC confirm safety without major adverse side effects.

The invention further provides a method of treatment of acute and chronic inflammatory diseases, including autoimmune diseases and/or of multiple system atrophy, multiple sclerosis, amyotrophic lateral sclerosis, and stroke, comprising administering the membranous particles according to the invention to a subject in need thereof. The invention further provides use of the membranous particles according to the invention for the manufacture of a medicament for use in treatment of acute and chronic inflammatory diseases, including autoimmune diseases, and/or of multiple system atrophy, multiple sclerosis, amyotrophic lateral sclerosis, and stroke.

The invention further provides a pharmaceutical composition comprising the particles according to the invention, and a pharmaceutically acceptable excipient such as a solvent, an anti-oxidant and/or a buffering agent.

The invention further provides the pharmaceutical composition comprising the particles according to the invention for use in immunosuppressive therapy. Said immunosuppressive therapy preferably is for the treatment of acute and chronic inflammatory diseases, including autoimmune diseases. Examples of inflammatory diseases that may be treated with the membranous particles according to the invention include acne, Addison's disease, asthma, celiac disease, prostatitis, glomerulonephritis, graft-versus-host disease, Hashimoto's disease, interstitial cystitis, lupus erythematosus, inflammatory bowel diseases such as Crohn's disease, pelvic inflammatory disease, psoriasis, rheumatoid arthritis, sarcoidosis, scleroderma, sepsis, Sjögren's syndrome, type 1 diabetes, transplant rejection, and vasculitis. Said pharmaceutical composition may also be used as a medicament in the treatment of multiple system atrophy, multiple sclerosis, amyotrophic lateral sclerosis, and stroke.

Said pharmaceutical composition preferably is for use in the treatment and prevention of transplant rejection.

The invention further provides a method of treatment of acute and chronic inflammatory diseases, including autoimmune diseases and/or of multiple system atrophy, multiple sclerosis, amyotrophic lateral sclerosis, and stroke, comprising administering a pharmaceutical composition according to the invention to a subject in need thereof. The invention further provides use a pharmaceutical composition according to the invention for the manufacture of a medicament for use in treatment of acute and chronic inflammatory diseases, including autoimmune diseases, and/or of multiple system atrophy, multiple sclerosis, amyotrophic lateral sclerosis, and stroke.

The invention further provides inactivated mesenchymal stem cells, or parts thereof, for use as a medicament. Inactivation may occur by any mechanism known in the art, including heat treatment, radiation such as ultra-violet radiation and ionizing radiation such as X-ray radiation, and/or chemical treatment. Mesenchymal stem cells are preferably inactivated by heat treatment, preferably by incubation in a temperature-regulated water bath at 45-55° C., preferably at about 50° C., preferably for a period from 10 minutes to 1 hour, preferably for a period of about 30 minutes.

The term “parts of inactivated mesenchymal stem cells”, as is used herein, refers to membranous parts that are obtained after inactivation of the stem cells. Said membranous parts comprise plasma membrane fragments.

Said inactivated mesenchymal stem cells, or parts thereof, preferably are for use as medicament in the treatment of acute and chronic inflammatory diseases and of autoimmune diseases, and/or of multiple system atrophy, multiple sclerosis, amyotrophic lateral sclerosis, and stroke.

Said inactivated mesenchymal stem cells, or parts thereof, preferably are for use as a medicament in the treatment and prevention of transplant rejection.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

### EXAMPLES

### Example 1

### Material and Methods

Isolation and Culture of MSC

Human MSC were isolated from subcutaneous adipose tissue that was surgically removed from the abdominal incision from healthy kidney donors. Adipose tissue was collected after written informed consent, as approved by the Medical Ethical Committee of the Erasmus University Medical Center Rotterdam (protocol no. MEC-2006-190). MSC were isolated from the adipose tissue as described previously (Roemeling-van Rhijn et al. 2012. Kidney Int 82: 748-758; Hoogduijn et al., 2007. Stem Cells Dev 16: 597-604). In short, the tissue was mechanically disrupted and washed with PBS. The adipose tissue was then digested enzymatically with 0.5 mg/mL collagenase type IV (Life Technologies, Paisley, UK) in RPMI 1640 Medium with glutaMAX (Life Technologies) for 30 min at 37° C. under continuous shaking. The stromal vascular fraction (SVF) was resuspended in minimum essential medium Eagle alpha modification (MEM-α; Sigma-Aldrich, St Louis, Mo., USA) containing 2 mM L-glutamine (Lonza, Verviers, Belgium), 1% penicillin/streptomycin solution (P/S; 100 IU/ml penicillin, 100 IU/ml streptomycin; Lonza). MSC were cultured in a 175-cm2 cell culture flask in MEM-α supplemented with 2 mM L-glutamine, penicillin/streptomycin (P/S) and 15% fetal bovine serum (FBS; Lonza) and kept at 37° C., 5% CO2 and 20% 02. Medium was refreshed once a week and MSC were passaged at around 80-90% confluence using 0.05% trypsin-EDTA (Life Technologies). To generate immune activated MSC, the cells were cultured for 3 days with 50 ng/ml IFNγ.

Preparation of Particles

MSC between passage 2-7 were used for particle preparation. Control MSC and MSC pre-cultured with IFNγ, were removed from the culture flasks by trypsinisation with 0.05% trypsin-EDTA. MSC suspensions were washed twice with PBS. The cells were then lysed in a hypertonic buffer or in H₂O and shaken vigorously for 5 minutes. The suspension was then centrifuged at 1000 g for 5 min to remove cellular debris. The collected supernatant was washed twice with isotonic buffer at 1000 g for 5 min. The supernatant was then centrifuged at 1500 g for 10 min. In the next step the supernatant was centrifuged at 100,000 g for 20 min in an ultracentrifuge. The pellet containing the membrane particles was reconstituted in isotonic buffer. The last step may be replaced by filtering the particles out of the suspension by use of Centricon Plus-70 Centrifugal Filter tubes (Ultracel-PL Membrane, 100 kD) (Merck Millipore) that separates the membrane particles from soluble proteins by centrifugation at 600 g.

Size Determination by NanoSight

Analysis of absolute size distribution of MSC membrane particles was performed using NanoSight NS300 (NanoSight Ltd., Cambridge, UK). Particle suspension (10 μl) was diluted in 1 ml of filtered PBS. The NanoSight settings were: temperature 23.25±0.5° C.; viscosity 0.92±0.01 cP, frames per second 25, measurement time 60 s.

Confocal Microscopy

Particles isolated from MSC that were labeled with fluorescent PKH-26 (Sigma Aldrich, St. Louis, Mo., USA) rendering the membranes fluorescent, were imaged on a Leica TCS SP5 confocal microscope (Leica Microsystems B.V., Science Park Eindhoven, Netherlands) equipped with Leica Application Suite—Advanced Fluorescence (LAS AF) software, DPSS 561 nm lasers, using a 60× (1.4 NA oil) objective. Optical single sections were acquired with a scanning mode format of 1,024×1,024 pixels and 8 bit/pixel images. Images were processed using ImageJ 1.48 (National Institutes of Health, Washington, USA).

Flow Cytometry

The immunophenotype, size and granularity parameters of MSC and MSC membrane particles were determined by FACS Canto II (BD Biosciences, San Jose, Calif.). MSC and MSC particles were incubated in PBS with CD73-PE, CD90-APC and PDL1-PE antibodies (all BD Biosciences) for 15 min at room temperature in the absence of light. The particles were not washed after staining to avoid loss of particles. MSC and particles were identified on the flow cytometer on the basis of their forward scatter (FSC) and side scatter (SSC) signals. The fluorescence signals were compared with unstained MSC or unstained particles.

CD14⁺ Monocyte Experiments

Human monocytes were isolated from PBMC by MACS sorting via positive selection for CD14⁺ with microbeads (Miltenyi, Bergisch Gladbach, Germany). CD14⁺ cells were incubated with various concentrations of MSC membrane particles or particles from IFNγ-treated MSC in RPMI medium (Life Technologies) and 10% heat inactivated FBS (30 min 57° C.) in non-adherent polypropylene tubes. After 24 h, monocytes were harvested and expression of CD90 and PDL1 determined by flow cytometry. Quantitative mRNA expression of IL6 and IL10 was determined by real-time RT-PCR using universal PCR master mix (Life Technologies) and assays-on-demand for IL-10 (Hs00174086.m1) and IL6 (Hs 00174131.m1) (Applied Biosystems, Foster City, Calif.) and analysed on an ABI PRISM 7700 sequence detector (Applied Biosystems). Data is expressed as relative copy number of the PCR products with respect to the housekeeping gene GAPDH. Relative copy number was calculated using the formula 2 (40-Ct value). Data was normalized to the controls (set at one).

In Vivo Administration of MSC Particles

C57BL6 mice received 5 mg/kg LPS (Sigma-Aldrich) via tail vein injections. One hour later the animals received 10×10⁹ MSC particles via the tail vein. Six hours after LPS injection the animals were sacrificed and blood collected in Minicollect EDTA tubes (Greiner Bio-One, Alphen a/d Rijn, Netherlands). Plasma was frozen at −80° C. and later used for measurement of cytokine/chemokine levels by multiplex assay (Merck Millipore, Billerica, Mass., USA) according to the manufacturer's manual.

### Results

Characterisation of MSC Membrane Particles

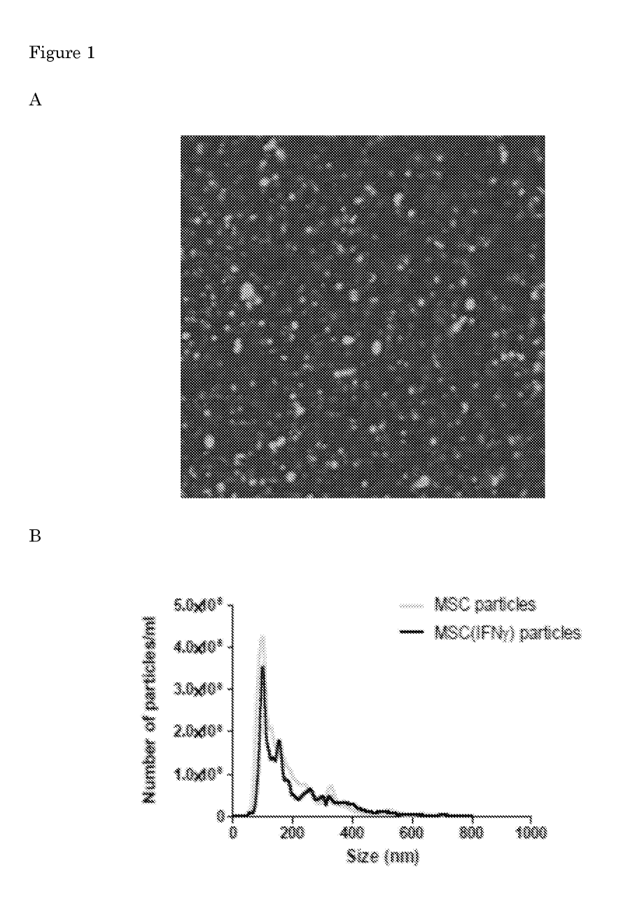

Exposure of MSC to hypotonic buffer resulted in lysis of MSC. Subsequent centrifugation at 1000 g and 1500 g separated organelles from membrane fragments and soluble factors. Centrifugation at 100,000 g separated the membrane fragments from soluble factors. The membrane fragments mostly appeared as round structures when observed by confocal microscope (FIG. 1A). The size distribution of the MSC membrane particles was determined by NanoSight. The size of the particles ranged from 70 nm to 600 nm, with a peak size distribution at 100-120 nm (FIG. 1B). There was no difference in size between particles from control MSC and MSC pre-treated with IFNγ.

Flow Cytometric Analysis of MSC Membrane Particles

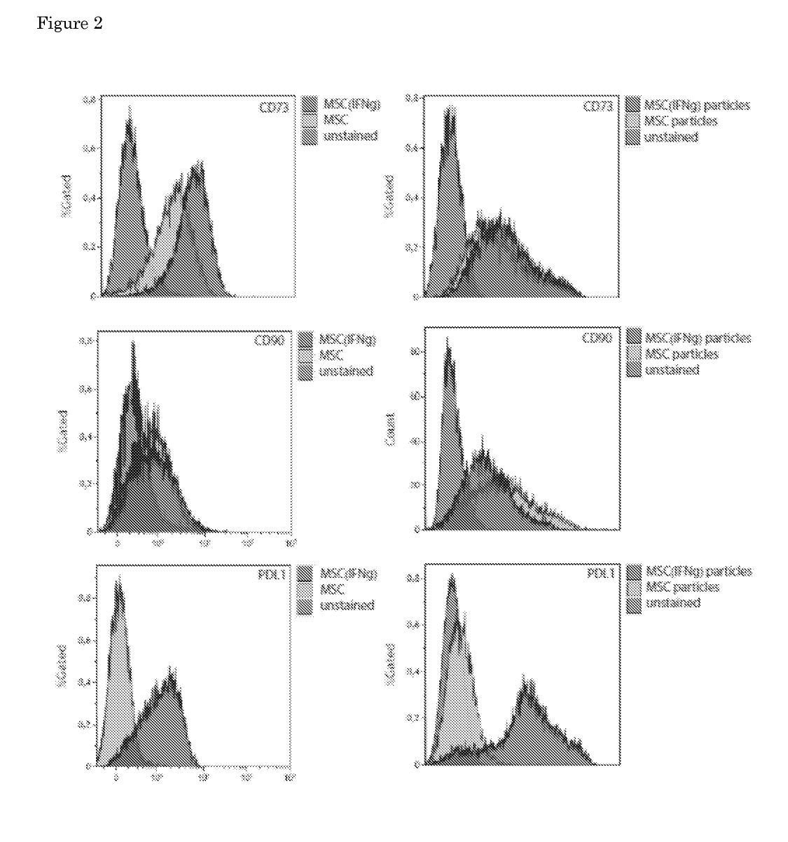

MSC and IFNγ treated MSC were immunophenotyped by flow cytometry. MSC and IFNγ treated MSC showed similar expression levels of the MSC surface markers CD73 and CD90 (FIG. 2 left panel). PDL1 was only expressed in MSC after treatment with IFNγ. Membrane particles mimicked the expression pattern of the MSC they were derived from. CD73 and CD90 were expressed on particles from both control MSC and IFNγ-treated MSC (FIG. 2 right panel). Membrane particles from IFNγ-treated MSC but not from control MSC contained PDL1.

Effects of MSC Membrane Particles on Monocytes

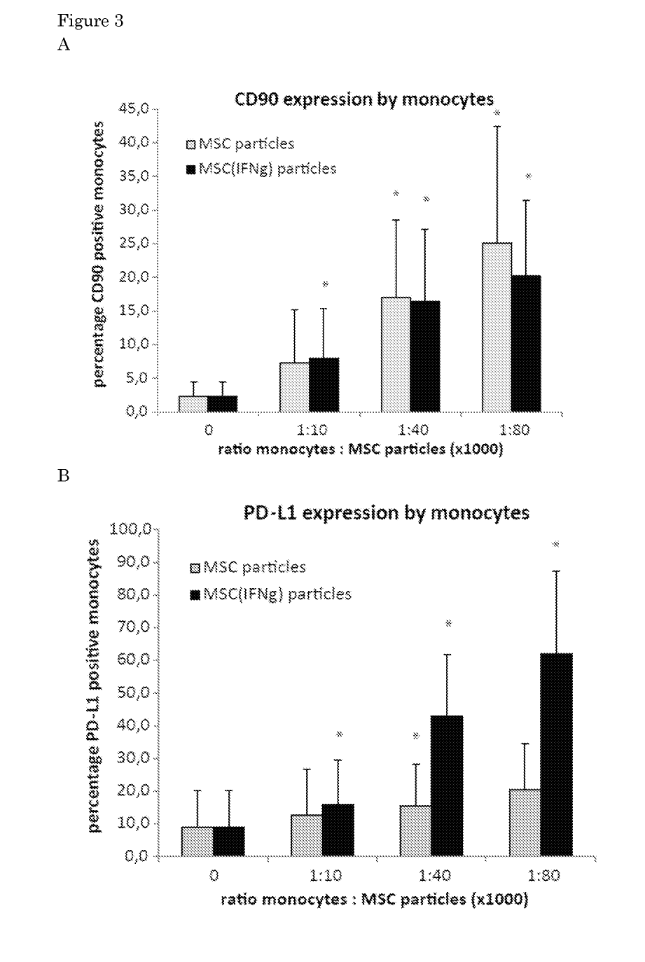

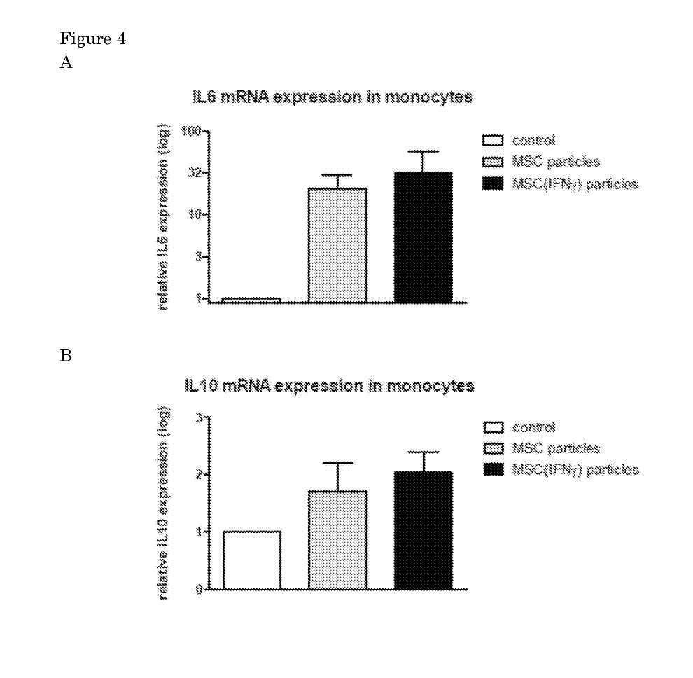

To investigate the effect of MSC membrane particles on human monocytes, CD14⁺ monocytes were isolated from PBMC and cultured in the presence of various concentrations of particles for 24 h. MSC and IFNγ-treated MSC membrane particles induced CD90 protein expression on monocytes in a dose-dependent fashion indicating activation of monocytes (FIG. 3A). Membrane particles from control MSC had no effect on anti-inflammatory PDL1 protein expression on monocytes. In contrast, membrane particles from IFNγ-treated MSC dose-dependently increased PDL1 expression on monocytes (FIG. 3B). As shown in FIG. 2, CD90 and PDL1 are also present on (IFNγ treated) MSC membrane particles and the expression of CD90 and PDL1 on monocytes could therefore represent transfer of protein or uptake of MSC membrane particles by monocytes. However, CD90 and PDL1 protein expression on monocytes was associated with mRNA expression for CD90 and PDL1 (data not shown). This indicates that MSC membrane particles induce gene expression changes in monocytes. This is further evidenced by increases in mRNA expression of immunomodulatory IL6 and IL10 in monocytes 24 h after incubation with membrane particles of MSC and IFNγ-treated MSC (FIG. 4).

Immunomodulatory Effects of MSC Particles In Vivo

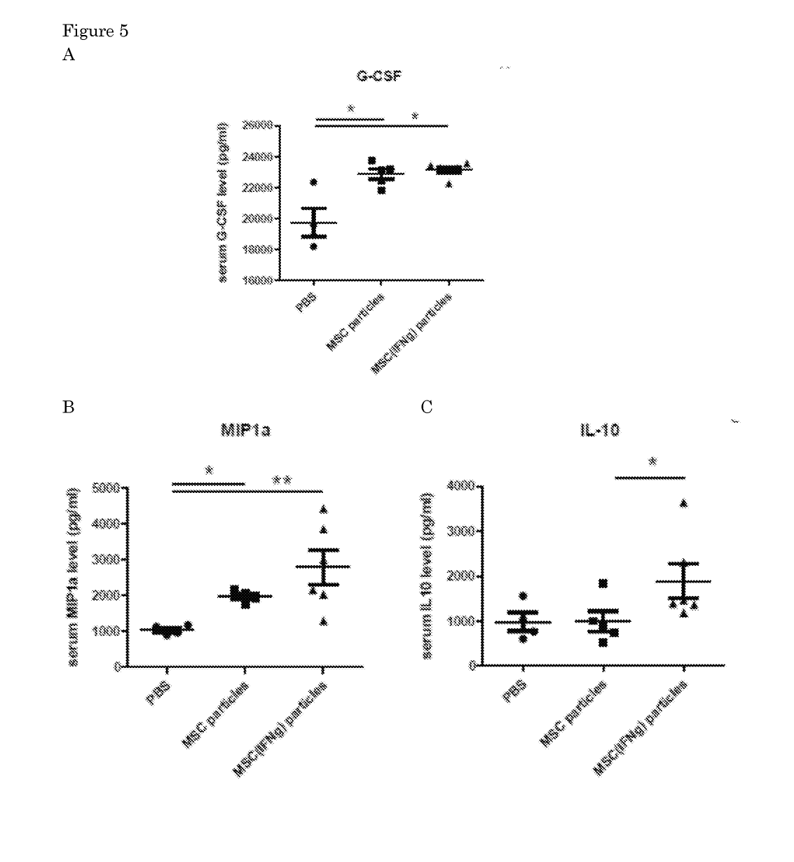

To examine the safety and immunomodulatory effects of MSC membrane particles in vivo, 10×10⁹ MSC particles or MSC(IFNγ) particles were injected via the tail vein in C57BL6 mice one hour after induction of sepsis-like systemic inflammation by LPS injection (5 mg/kg). MSC particles were well tolerated by the animals and no adverse effects were observed. Both MSC particles and MSC(IFNγ) particles induced a systemic immunomodulatory response, demonstrated by increases in serum levels of G-CSF and MIPla 5 hours after particle infusion (FIGS. 5A and B). MSC(IFNγ) particles, but not MSC particles, increased serum IL10 levels (FIG. 5C), indicative for an anti-inflammatory response.

### Example 2

### Material and Methods

Materials and methods were as described in Example 1, except when indicated otherwise.

Transmission electron microscopy examination of MSC particles MSC particles (MP) were fixed with 2% paraformaldehyde and adsorbed onto carbon-coated grids for 5 min. The grids with adherent MPs were washed in milliQ water for 1 min. For negative staining, the grids were floated on drops of uranyl acetate for x min. The excess of liquid was blotted manually from the edge of the grids. The grids were analyzed in a Tecnai Spirit microscope (EM) (FEI, The Netherlands) equipped with a LaB6 cathode. Images were acquired at 120 kV and room temperature with a 1376×1024 pixel CCD Megaview camera.

ATPase Assay

ATPase activity from MP and MPγ was measured using an ATPase assay kit according to the manufacturer's instructions (Sigma-Aldrich). A phosphate standard was used for creating a standard curve. MP (1×1012, 1×1011, 1×1010, 1×109/ml) were incubated with 4 mM ATP for 30 min at room temperature in assay buffer with malachite green reagent. The formation of the colorimetric product that formed in the presence of free phosphates was measured with a spectrophotometer at 620 nm.

As a control for possible phosphate contamination, the four MP concentrations were incubated in assay buffer without ATP. The signal from these samples was subtracted from the samples incubated with ATP.

CD73 Activity Assay

A modified protocol of CD73 inhibitor screening assay kit (BPS Bioscience) was used to determine whether MP are able to degrade AMP into adenosine plus phosphate. MP and MPγ (1×10¹², 1×10¹¹, 1×10¹⁰/ml) were incubated with AMP (500 μM) during 25 minutes at 37° C. Then, colorimetric detection reagent was added to measure the free phosphate from the CD73 reaction. Samples without AMP were measured as a control for free phosphate contamination. CD73 enzyme (2 and 1 ng) was used to calculate the concentration of CD73 in the MP, and MPγ.

Esterase Activity by CFSE

CFDA-SE, which is non-fluorescent, enters the cytoplasm of cells, intracellular esterases remove the acetate groups and convert the molecule to the fluorescent ester (CFSE). This application was used to detect whether MP have esterase activity. After MP generation, 1×10¹⁰ particles/ml were labeled with 50 μM of CFDA-SE and incubated at 37° C. during 30 min. Several dilutions were performed (1×10⁹, 1×10⁸, 1×10⁷ particles/ml) to obtain a proper stoichiometry of CFSE staining. As control, PBS, PBS+CFDA-SE, and non-stained MP were used. CFSE fluorescence was measured by flow cytometry (FACS Canto II, BD Biosciences). Due to the small size of the MP, reliable FSC and SSC measurements could not be obtained. Instead, MP were identified by setting a fluorescence threshold triggering on the FITC channel so that events above the threshold could be identified as CFSE-loaded MP.

CD3/CD28 T Cell Proliferation Assay

To evaluate the immunomodulatory capacity of MP, PBMC were labeled with 1 μM of CFSE and plated in round bottom 96-well culture plates at a density of 5×104 cells/well. T cell proliferation was stimulated by adding human anti-CD3/anti-CD28 antibodies (1 μl/ml each) with a linker antibody Ig (2 μl/ml) (BD Biosciences). PBMC were incubated with different ratios of MP, and MPY (1:5.000, 1:10.000, 1:40.000, 1:80.000) for 4 days. On the fourth day, non-adherent PBMC were removed from the plate, washed with FACS Flow and incubated with monoclonal antibodies against CD4-PerCP and CD8-PE-Cy7 (antibodies were purchased from BD Biosciences) at room temperature for 30 minutes. After washing with FACS Flow, the samples were analyzed by flow cytometry.

MP Uptake Assays

To obtain fluorescent MP, MSC were labeled with the red fluorescent chromophore PKH-26 dye, which intercalates into lipid bilayers, according to the manufacturer's instructions (Sigma-Aldrich). Then, MP from PKH-26 labeled MSCs were generated (PKH-MP).

Human PBMC from healthy donors were isolated by density gradient centrifugation (Ficoll Isopague, Sigma Aldrich) and cultured with PKH-MP (ratio 1:40000). The incubation conditions were 37° C., 5% CO2, and 95% humidity. As a control of the uptake process, PBMC were incubated with PKH-MP at 4° C. PKH-MP uptake by lymphocytes and monocytes was analyzed by flow cytometry (FACS Canto II, Becton Dickinson) at 1 h, and 24 h.

Confocal microscopy analysis of PKH-MP uptake by monocytes was carried out by isolating CD14⁺ cells from PBMC using auto-MACS Pro by positive-selection (Miltenyi Biotec, Leiden, The Netherlands). Then, monocytes were labelled with luM of CFSE (Life Technologies) and cultured with PKH-MP (ratio 1:4×10⁴). Time-lapse images of monocytes were performed on a Leica TCS SP5 confocal microscope (Leica Microsystems B.V., Science Park Eindhoven, Netherlands) equipped with Leica Application Suite—Advanced Fluorescence (LAS AF) software, DPSS 561 nm lasers, using a 60× (1.4 NA oil) objective. The microscope was equipped with a temperature-controlled incubator. The temperature was maintained at 37° C., and the CO2 at 5%. Images were processed using ImageJ 1.48 (National Institutes of Health, Washington, USA).

Statistical Analysis

Data were analyzed using paired t-test or Wilcoxon signed-rank test depending on the distribution of the data as tested with Kolmogorov-Smirnov test for normality using GraphPad Prism 5 software. Parametric data are expressed as means, whereas nonparametric data are expressed as medians. A value of P<0.05 was considered statistically significant. Two-tailed P values are stated.

### Results

Generation and Characterization of MP

MP were generated from unstimulated and IFN-γ stimulated MSC. The number of cells used for each analysis was between 1×10⁶-1.5×10⁶ cells (80% confluency). Based on the particle concentration per ml, the average number of particles generated from each MSC was 1.2×10⁶±2.7×10⁵ for MP and 1.1×10⁶±2.8×10⁵ for MPγ. There was no significant difference in size distribution or concentration (particles/MSC) between MP and MPγ.

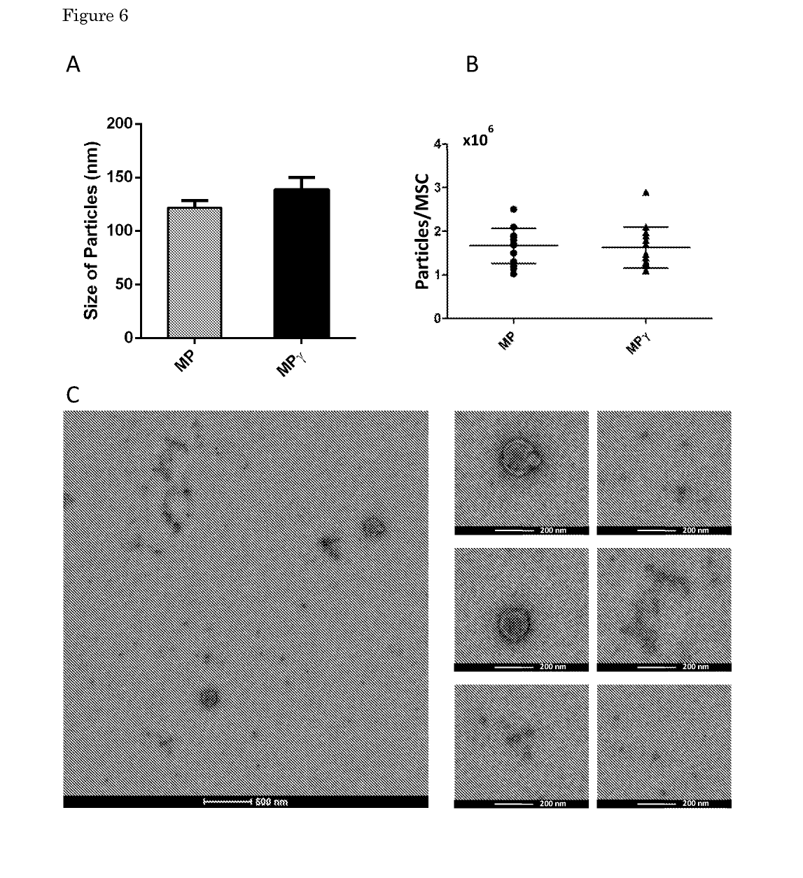

Transmission electron microscopy analysis confirmed the NTA results. Most of the MP had a size <200 nm (FIG. 6), but also larger particles were found. MP showed a spherical shape.

MP Possess Enzyme Activity

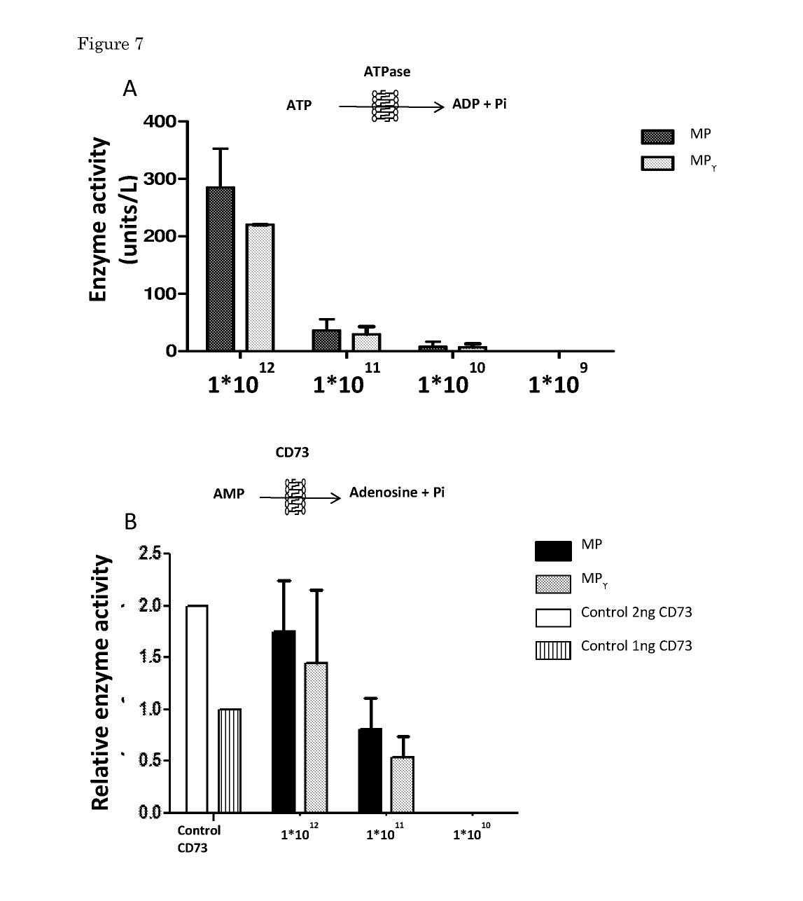

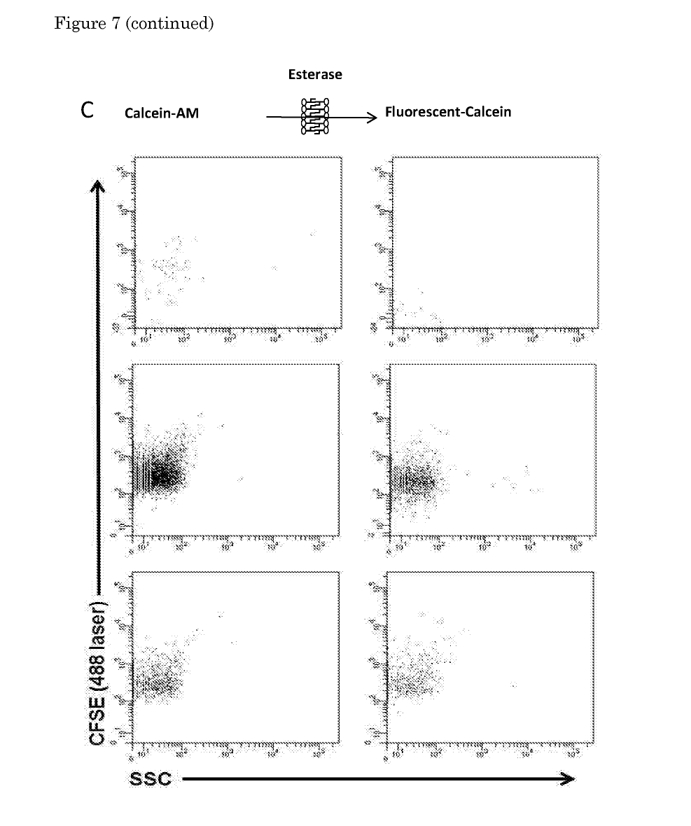

To analyze whether the MP have enzyme activity, we examined the ability of MP, and MPγ to convert ATP to ADP by ATPase activity, and AMP to adenosine by the nucleotidase activity of CD73. The last product of these two reactions is free phosphate, so the samples for these assays were prepared with milliQ water to avoid the contamination with free phosphates from the saline buffers. FIG. 7A shows the ATPase activity (units/1) calculated from the standard curve generated with known free phosphate concentrations. MP and MPγ were able to convert ATP to free phosphate and the level of free phosphate was dependent on the concentration of MP. There was no statistical difference between MP and MPγ. To examine whether MP, and MPγ possess CD73 activity, the production of free phosphates by 2, and 1 ng of purified CD73 was compared with different concentrations of MP, and MPγ. Both types of MP were able to produce free phosphates after adding the substrate (AMP). The detection of free phosphate was dependent on concentration of MP and the amount of CD73 present in MP was calculated through the CD73 controls (FIG. 7B).

Esterase activity was measured by the conversion of CFDA-SE to CFSE by flow cytometry based on fluorescence triggering strategy (FIG. 7C). Fluorescent particles were not detectable in the controls PBS, PBS+CFSE, and non-labeled MP. When the MP were labeled with CFSE (CFSE-MP), fluorescent events were observed. The number of CFSE-MP detected was dependent on concentration of MP in the samples. Furthermore, the fluorescence intensity of the MP did not decrease when the samples were diluted. This fact means that single MP can be detected with the FACS strategy. Furthermore, the FACS analyses demonstrate that the esterase activity is related to the presence of MP.

Effects of MP on PBMC Proliferation

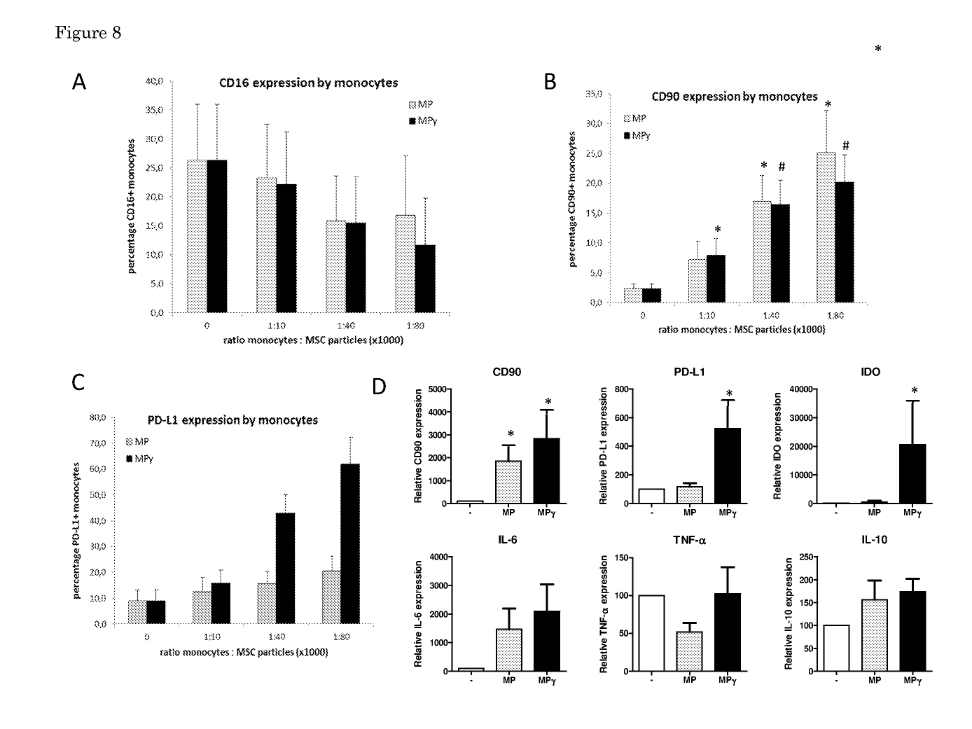

PBMC stimulated with anti-CD3/antiCD28 were cultured with different ratios of MP for 4 days (1:5.000, 1:10.000, 1:40.000, 1:80.000). Addition of MP or MPγ did not affect the proliferation of CD4+ and CD8+ T cells (data not shown). MP decrease the proportion of CD16⁺ monocytes and increase CD90+ and PD-L1⁺ monocyte subsets Monocytes were cultured with different ratios of MP for 24 h (1:10.000, 1:40.000, 1:80.000) to determine whether MP could affect monocyte cell surface markers expression and immune function. Monocytes were cultured in polypropylene tubes to avoid the adherence of the cells and differentiation into macrophages. Culture of monocytes in the presence of MP or MPγ treatment decreased the frequency of pro-inflammatory CD14⁺ CD16+ cells at ratios of 1:40.000 (by 45% and 49%, respectively) and 1:80.000 (by 48% and 35%, respectively) (FIG. 8A). Monocytes treated with MP at ratios of 1:40.000 and 1:80.000 furthermore increased the expression of CD90 by 17% and 25%, respectively. Meanwhile, the MPγ group showed an increase in CD90 expression at ratios of 1:10.000 by 8%, 1:40.000 by 16% and 1:80.000 by 20% (FIG. 8B). Moreover, MPγ treatment induced anti-inflammatory PD-L1 expression in monocytic cells by 16% at a 1:10.000 ratio, 43% at a 1:40.000 ratio and 62% at a 1:80.000 ratio. MP had a smaller effect on PD-L1 expression with a 15% increase at a ratio of 1:40.000 (FIG. 8C).

MP Affect the Expression of Pro- and Anti-Inflammatory Genes in Monocytes

In order to further examine the effect of MP on monocyte immune function, and to examine whether the immunophenotypic changes observed were a result of protein transfer or of gene expression regulation, mRNA expression of a number of genes with pro- and anti-inflammatory function was analyzed in monocytes by qPCR after 24 h of stimulation with MPs. Upregulation in CD90 gene expression as a result of particles stimulation was observed in MP and MPγ treated monocytes (p<0.05) (FIG. 8D). Moreover, expression of the anti-inflammatory factors IDO and PD-L1 was increased in monocytes treated with MPγ, but not MP (p<0.05) (FIG. 8D). There was a trend for increased expression of IL-6 after MP and MPγ treatment, but this was not significant. Significant changes in gene expression were also not observed for the pro-inflammatory cytokinesTNF-α and anti-inflammatory cytokine IL-10.

Monocytes but not Lymphocytes are Able to Uptake MP

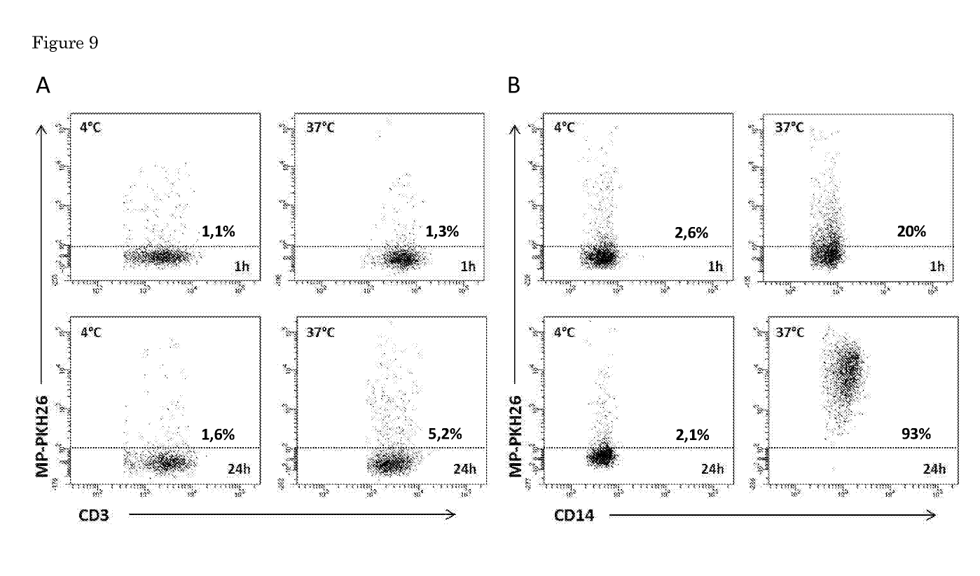

Since the previous results showed that MP had immunomodulatory properties on monocytes but not on lymphocytes we analyzed the interaction of MPs with both types of immune cells. With that purpose PKH-MP were added to PBMC (ratio 1:40,000) and incubated during 1 h, and 24 h at 37° C. As control the cells were incubated at 4° C., at which temperature no active uptake of MP is expected. 1 h after the addition of MP, a small percentage of CD3-lymphocytes (1.3±0.2%) were positive for PKH-MP (FIG. 9A) while 20±5.3% of CD14-monocytes was able to uptake MP (p<0.05) (FIG. 9B). The difference between the MP uptake by monocytes and lymphocytes was higher after 24 h (lymphocytes: 5.2±1.4%, monocytes: 93±4.3%; p<0.05). The 4° C. control for uptake was always below 3% for monocytes and lymphocytes in all the time points. This result indicated that MP uptake was mediated in an energy-dependent process.

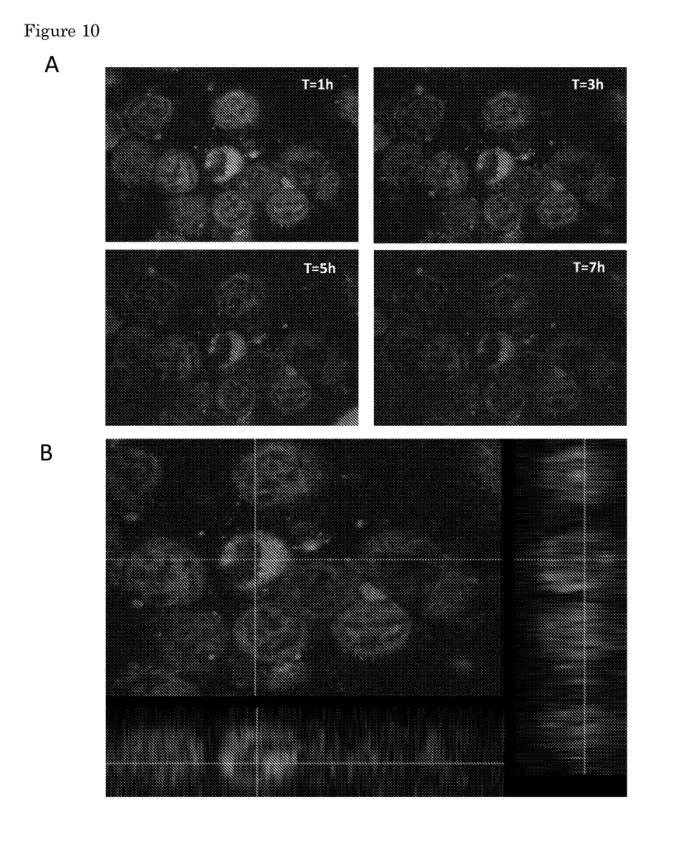

To examine whether MP could be internalized by the monocytes, confocal immunofluorescence microscopy was performed with isolated CD14 cells from PBMC. Monocytes were labeled with CFSE and cultured with PKH-MP (1:40,000). Time-lapse recordings showed that MP bound to the plasma membrane of the monocytes but they were not internalized (FIG. 10A). To look in detail at the localization of MP on the monocytes, z-stack images were analyzed by confocal microscopy (FIG. 10B). These images confirmed that MP remained localised to the cell surface of the monocytes.

1-15. (canceled)

16. Immunomodulatory particles from lysed mesenchymal stem cells comprising membranous structures from plasma membrane of said mesenchymal stem cells, said membraneous structures comprising immunosuppressive compounds.

17. The immunomodulatory particles according to claim 16, wherein the immunosuppressive compounds are selected from steroids, cytostatics, antibodies, and/or calcineurin inhibitors.

18. A method of treatment comprising
administering immunomodulatory particles from lysed mesenchymal stem cells comprising membranous structures from the plasma membrane of said mesenchymal stem cells to a subject in need thereof, to thereby treat said subject.

19. The method according to claim 18, wherein the particles have an average particle size of between 70 and 170 nm.

20. The method according to claim 18, whereby the mesenchymal stem cells were treated with interferon gamma, prior to their lysis.

21. The method according to claim 18, whereby the mesenchymal stem cells are isolated from adipose tissue.

22. The method according to claim 18, wherein the subject suffers from acute and chronic inflammatory diseases, including autoimmune diseases.

23. The method according to claim 18, wherein the subject suffers from transplant rejection.

24. The method according to claim 18, wherein the mesenchymal stem cells are obtained from the subject that is treated with said particles.

25. A pharmaceutical composition comprising immunomodulatory particles from lysed mesenchymal stem cells comprising membranous structures from the plasma membrane of said mesenchymal stem cells, and a pharmaceutically acceptable excipient.

26. The pharmaceutical composition according to claim 25, wherein the particles comprise immunosuppressive compounds, preferably steroids, cytostatics, antibodies, and/or calcineurin inhibitors.

27. Immunosuppressive therapy, comprising the administration of the pharmaceutical composition according to claim 25.

28. A method of treatment or prevention of transplant rejection, comprising administering the immunosuppressive therapy according to claim 27.

29. A method of treatment comprising
administering inactivated mesenchymal stem cells, or parts thereof, to a subject in need thereof, to thereby treat said subject.

30. The method of treatment according to claim 29, comprising
administering inactivated mesenchymal stem cells, or immunomodulatory particles comprising membranous structures from the plasma membrane of said inactivated mesenchymal stem cells, to a subject for the treatment of acute and chronic inflammatory diseases, including autoimmune diseases and/or for prevention of transplant rejection.
